(19) United States
(12) Patent Application Publication
(10) Pub. No.: US 2007/0134309 A1
(43) Pub. Date: Jun. 14, 2007

(54) Agent containing ergoline for transdermal application

(76) Inventors: Reinhard Horowski, Berlin (DE); Johannes Tack, berlin (DE); Katalin Tisa Bostedt, pullach (DE); Dirk Schenk, Dietramszell (DE)

Correspondence Address: PILLSBURY WINTHROP SHAW PITTMAN, LLP, P.O. BOX 10500, MCLEAN, VA, 22102, US

(21) Appl. No.: 10/570,262

(22) PCT Filed: May 30, 2004
(86) PCT No.: PCT/DE04/01133

§ 371 Date: Oct. 30, 2006

(30) Foreign Application Priority Data
Sep. 3, 2003 (DE) 103 41 317.0

(51) Int. Cl. A61K 9/70

(52) U.S. Cl. 424/449

(57) ABSTRACT

The invention relates to a transdermal therapeutic system containing ergolin compounds, preferably lisuride, in which the ergolin compound is stabilized. The oxidation-sensitive ergolin compound is stabilized by a combination of at least one liposoluble, free radical-scavenging antioxidant, preferably di-tert.-butyl methyl phenol, di-tert.-butyl metoxy phenol, tocopherol or ubiquinone and a basic polymer.

The present application is a national stage application under 35 U.S.C. §371 of PCT/ DE2004/001133 filed May 30, 2004, which claims priority to German Application No. DE10341317 filed Sep. 3, 2003. The entirety of these applications are hereby incorporated by reference.

### FIELD OF THE INVENTION

The present invention relates generally to a transdermal therapeutic device and system for delivering a stabilized ergoline derivative compound. More specifically, the present invention relates to an extended shelf-life transdermal therapeutic device for delivering an ergoline derivative compound transdermally.

Some transdermal therapeutic systems containing ergoline derivatives are used for treating diseases caused by disorders of the dopaminergic system (See, for example, WO 92/20339, WO 91/00746).

Transdermal therapeutic systems with oxidation sensitive active ingredients are not very stable. One method for improving the stability of a system with oxidation sensitive active ingredients is described in DE 100 54 713 A1, in which all formulation components of the system are selected so that the total of their peroxide numbers (as a measure for oxidizability) does not exceed 20. However this implies a restriction as to the ingredients that qualify or a great pre-treatment effort of each ingredient with sodium hydrogen sulfite solutions to destroy the existing peroxides.

### DETAILED DESCRIPTION OF THE INVENTION

The invention relates to an agent for transdermal application consisting of an impermeable top layer, a matrix containing ergoline compounds and, optionally, penetration-enhancing agents, an optional diffusion barrier covering the matrix, an adhesive layer that is permeable to these substances, and a peelable protective layer. The ergoline derivatives, particularly lisuride, in transdermal therapeutic systems are to be stabilized.

Transdermal therapeutic systems containing ergoline derivatives are used for treating diseases caused by disorders of the dopaminergic system. Also, they seem particularly suited for treating Parkinson's disease, Parkinsonism, restless legs syndrome, for the prophylaxis of premenstrual syndrome, and as an agent for inhibiting lactation. They are sometimes used in migraine prophylaxis, which also requires a well tolerable and constant therapy.

Transdermal therapeutic systems may be designed as so-called matrix systems. The matrix systems typically consist of an impermeable top layer, a matrix in which the active agent formulation is embedded or dissolved, and—unless the matrix is adhesive itself—of an adhesive layer and a peelable protective layer.

The active agent is typically combined with appropriate adjuvants such as solvents, penetration-enhancers and crystallization inhibitors to achieve a defined and uniform flux.

But the problem with prior art preparations of ergoline derivatives is the instability of the active agent itself. Transdermal therapeutic systems with ergoline derivatives can show discoloration, typically associated with a decline in active agent concentration, after some time. This is because ergoline derivatives have considerable sensitivity to oxidation. Lisuride, for example, is oxidized on the nitrogen in position 6 of the ring system even without any exposure to light.

This causes skin irritation, in particular when used over a longer period of time. As the decline in concentration of the active agent is unknown, controlled dosage can no longer be ensured.

The antioxidants typically used as stabilizers such as citric acid, ascorbic acid, sodium sulfite, sodium disulfite, alkyl gallate, ascorbyl palmitate and the like do not result in any substantial improvement.

It is the object of this invention to provide a transdermal therapeutic system containing an ergoline derivative that is resistant to storage and in which decomposition of the active agent is prevented so that it can stay on the skin for longer periods of time without causing irritation.

This object is achieved according to the invention in that ergoline derivatives in a transdermal therapeutic system are stabilized by a combination of at least one liposoluble, free radical-scavenging antioxidant, preferably di-tert-butyl methyl phenolene, di-tert-butyl methoxyphenolene, tocopherol, or ubiquinone and a basic polymer.

Studies have shown that the presence of one of the above mentioned antioxidants alone does not result in any significant improvement of the stability of the ergoline derivatives.

Transdermal therapeutic systems, in which a basic polymer such as a butyl methacrylate-(2-dimethylaminoethyl)methacrylate-methyl methacrylate copolymer (Eudragit E 100 by Rohm, Germany) is present in addition to the antioxidants mentioned above, show surprisingly great stability. The basic polymer may also be present in a mixture with other common polymers such as neutral polyacrylates. In addition, the polymer mixture may contain common tackifiers (such as resins or polyacrylates) to improve adhesion.

The systems of the invention typically have a weight per unit area of 2 to 10 mg/cm². This results from the total of all ingredients after drying. The content of matrix-forming polymers is 50 to 95% by wt., preferably 60 to 85%. Other polymers make up from 5 to 30% by wt., preferably from 10 to 20%. Antioxidant content is between 0.25 and 5% by wt., preferably from 0.5 to 1.5%. The active ingredient content is at 1 to 10% by wt., preferably at 3 to 6%.

The combinations according to the invention have an unexpected synergy effect that hampers oxidation of ergoline derivatives in transdermal systems.

### Exemplary Embodiments

### EXAMPLE I

Stability studies were performed using samples that contained combinations of various antioxidants and polymers.

The active agent used was lisuride. The samples further contained other adjuvants that are commonly used in transdermal therapeutic systems.

Sample Preparation:

150 g of polyvidone and 300 g of dibutyl sebacate as softeners and 20 g of Floral E 105 (hydrated colophonium pentaerthrite ester by Hercules) as a tackifier are successively added at room temperature under stirring to 900 g of an approx. 50% solution of polymer adhesive in a mixture of 2-propanol and acetone. Then, 50 g of lisuride and 15 g of antioxidant are presuspended in a portion of the solvent and added to the adhesive solution under continuous stirring. When the ingredients have completely dissolved, the solution is brought to its final weight using acetone and allowed to stand for approx. 24 hours to remove the gas bubbles. The solution is subsequently applied to a siliconized polyester film (liner film) using a suitable coating device (such as knife-over-roll) so that a uniform film with a weight per unit area of approx. 5 mg/cm² is formed after removal of the volatile solvents at 40 to 90° C. The product is then coated with a top film made of polyester. The laminate thus obtained is divided into individual patches with an area of 10 cm² using a suitable punching device and placed in opaque bags made of an aluminum/paper composite film.

Table 1 shows the composition of the samples studied.

TABLE 1
Sample compositions in percent by weight

| | Sample number | | | | | | | | | | |
|---|---|---|---|---|---|---|---|---|---|---|---|
| | #80 | #81 | #82 | #83 | #84 | #85 | #86 | #87 | #88 | #90 | #98 |
| Lisuride | 3.3 | 3.2 | 4.0 | 4.0 | 4.0 | 4.0 | 4.0 | 4.0 | 4.0 | 3.0 | 3.0 |
| MA24A¹⁾ | 44.9 | 45.1 | — | — | — | — | — | — | — | 53.0 | 52.0 |
| Eudragit E | — | — | — | — | 85.0 | 85.0 | 68.0 | 68.0 | 60.0 | — | — |
| 100²⁾ | | | | | | | | | | | |
| Durotac DT | — | — | 77.0 | 77.0 | — | — | 17.0 | 17.0 | 15.0 | — | — |
| 387-2510³⁾ | | | | | | | | | | | |
| Ascorbyl | — | — | — | — | — | — | — | — | — | — | 2.0 |
| palmitate | | | | | | | | | | | |
| Tocopherol | 1.0 | — | 1.0 | — | 1.0 | — | 1.0 | — | 1.0 | 1.0 | — |
| BHT⁴⁾ | — | 0.9 | — | 1.0 | — | 1.0 | — | 1.0 | — | — | — |
| Polyvidone | 9.2 | 9.3 | 10.0 | 10.0 | 10.0 | 10.0 | 10.0 | 10.0 | 20.0 | 10.0 | 10.0 |
| Transcutol⁵⁾ | 27.0 | 27.0 | — | — | — | — | — | — | — | 25.0 | 25.0 |
| Eutanol⁶⁾ | 8.7 | 8.6 | 5.0 | 5.0 | — | — | — | — | — | 8.0 | 8.0 |
| Dimethyl | 5.9 | 5.9 | — | — | — | — | — | — | — | — | — |
| acetamide | | | | | | | | | | | |

¹⁾Polyisobutylene by Adhesives Research Ltd., Ireland
²⁾Butyl methacrylate-(2-diaminoethyl)methacrylate-methacrylate copolymer (1:2:1) by Röhm, Germany
³⁾Neutral polyacrylate by National Starch, United States
⁴⁾Butylhydroxy toluene (2,6-di-tert-butyl-4-methylphenol)
⁵⁾Diethylene glycol monoethyl ether by Gattefossé, France
⁶⁾2-hexyl decanol by Cognis, Germany

Storage:

The samples were stored under the following conditions:
a) at 4° C.,
b) at 25° C and 60% humidity,
c) at 40° C. and 75% humidity.

The concentration of aminoxide obtained by oxidizing nitrogen at position 6 of the ergoline ring system (lisuride-N-oxide) was determined after one month of storage.

Determination of Aminoxide Content:

The quantity of aminoxide was determined using a HPLC method comprising the following parameters:
Column: Luna C18(II), 100 mm×4, 6 mm ID
Precolumn: Phenomenex C18, 4 mm×3 mm ID
Column temperature: 35° C.
Running time: 30 mins
Flow rate: 1.20 ml/min
Mobile phase: A : 10 mM TRIS buffer, pH 8.7 B: Acetonitrile
Gradient profile: 0 to 25th minute: 12% B 25^{th} to 27^{th} minute: 42% B 28^{th} to 38^{th} minute: 12% B
Detection: fluorescence detector
   Sample Preparation:

A lisuride patch produced as described in Example 1 is weighed and, after removing the liner film, shaken in 50 ml of solvent (2-propanol) for 15 minutes. 5 ml of the solution are then diluted to 20 ml using Diluent (acetonitrile). About 2 ml of this solution are centrifuged for 2 minutes at 5,000 rpm, and the clear supernatant solution is filled into a HPLC sample vial.

Table 2 shows the results.

TABLE 2
Formation of aminoxide from lisuride after one month of storage

| | Lisuride-N-oxide content in % by wt | | |
|---|---|---|---|
| Sample | a: 4° C. | b: 25° C. | c: 40° C. |
| #80: MA24A/Tocopherol | 0.51 | 1.59 | 2.80 |
| #81: MA24A/BHT | 0.45 | 1.26 | 1.86 |
| #82: Durotac/Tocopherol | 0.70 | 1.21 | 1.49 |
| #83: Durotac/BHT | 0.71 | 1.08 | 1.44 |
| #84: Eudragit/Tocopherol | 0 | 0.11 | 0.26 |
| #85: Eudragit/BHT | 0.06 | 0.09 | 0.22 |
| #86: Eudragit/Durotac/Tocopherol | 0 | 0.14 | 0.37 |
| #87: Eudragit/Durotac/BHT | 0 | 0.14 | — |
| #88: Eudragit/Durotac/Tocopherol | 0.11 | 0.21 | 0.44 |
| #90: MA24A/Tocopherol | — | — | 1.93 |
| #98: MA24A/ascorbyl palmitate | 0.27 | 0.58 | — |

### EXAMPLE 2

Stability studies were performed using samples that contained combinations of various antioxidants and basic polyacrylates.

The active agent used was lisuride. The samples further contained other adjuvants that are commonly used in transdermal therapeutic systems.

Sample Preparation:

175 g of polyvidone and 310 g of dibutyl sebacate as softeners and 175 g of dodecanol as cosolvent are added successively under stirring at room temperature to 1800 g of an approx. 45% solution of basic polyacrylate adhesive in acetone. Then, 80 g of lisuride and 17 g of antioxidant are presuspended in a portion of the solvent and added to the adhesive solution. 35 g of Floral are added as a tackifier after the ingredients have dissolved completely. The solution is brought to its final weight using acetone and allowed to stand for approx. 24 hours to remove the gas bubbles. The solution is subsequently applied to a siliconized polyester film (liner film) using a suitable coating device (such as knife-over-roll) so that a uniform film with a weight per unit area of approx. 5 mg/cm² is formed after removal of the volatile solvents at 40 to 90° C. The product is then coated with a top film made of polyester. The laminate thus obtained is divided into individual patches with an area of 10 cm² using a suitable punching device and placed in opaque bags made of an aluminum/paper composite film.

Table 3 shows the composition of the samples.

TABLE 3
Sample compositions in percent by weight

| | Sample number | | | | |
|---|---|---|---|---|---|
| | #C005 | #151 | #156 | #C001 | #152 |
| Lisuride | 5.0 | 5.0 | 5.0 | 4.0 | 5.0 |
| Polyvidone | 10.0 | 10.0 | 10.0 | 10.0 | 10.0 |
| Lauryl alcohol | 15.0 | 15.0 | 15.0 | — | 15.0 |
| Foral 105 E¹⁾ | 2.0 | 2.0 | 2.0 | — | 2.0 |
| BHT²⁾ | — | — | 1.0 | 0.4 | — |
| Sodium sulfite | — | — | — | — | 0.1 |
| Eudragit E 100³⁾ | 68.0 | 68.0 | 67.0 | 85.6 | 67.9 |

¹⁾hydrated colophonium pentaerythrite ester by Hercules
²⁾Butylhydroxy toluene
³⁾Butyl methacrylate-(2-diaminoethyl)methacrylate-methacrylate copolymer (1:2:1) by Röhm, Germany

Storage:

The samples were stored at 25° C. and 60% humidity and at 40° C. and 75% humidity. The aminoxide concentration was determined after 1 and 3 months of storage.

Determination of Aminoxide Content:

The aminoxide content was determined using the HPLC method described in Example 1.

Table 4 shows the results of the stability tests.

TABLE 4
Formation of aminoxide from lisuride after
one month and three months of storage

| | Lisuride-N-oxide content in % by wt | | | |
|---|---|---|---|---|
| | 25° C. | 25° C. | 40° C. | 40° C. |
| Sample | 1 month | 3 months | 1 month | 3 months |
| #C005: Eudragit | 0.20 | 0.50 | 1.18 | 3.51 |
| #151: Eudragit | 0.21 | 0.40 | 0.91 | 2.43 |
| #153: Eudragit | 0.21 | 0.48 | 0.92 | 2.00 |
| #156: | 0.14 | — | 0.27 | — |
| Eudragit/BHT | | | | |
| #C001: | 0.10 | 0.14 | 0.38 | 0.44 |
| Eudragit/BHT | | | | |
| #152: Eudragit/ | 0.18 | 0.36 | 0.82 | 2.23 |
| Natriumsulfite | | | | |

1. An agent for transdermal application consisting of an impermeable top layer, a matrix containing ergolin compounds and, optionally, penetration-enhancing agents, an optional diffusion barrier covering the matrix, an adhesive layer that is permeable to these substances, and a peelable protective layer, characterized in that the ergolin compounds are stabilized by an antioxidant and a basic polymer.

2. The agent according to claim 1 wherein the antioxidant is a compound that reacts with free radicals.

3. The agent according to claims 1 and 2 wherein the antioxidant is selected from di-tert.-butyl methyl phenols, tert.-butyl methoxyphenols, tocopherols, and/or ubiquinones.

4. The agent according to claims 1 to 3 wherein the antioxidant is present in quantities from 0.25% by wt. to 5% by wt.

5. The agent according to claims 1 to 4 wherein the basic polymer is an acrylate copolymer.

6. The agent according to claims 1 to 5 wherein the acrylate copolymer is a butyl methacrylate-(2-diaminoethyl)methacrylate-methacrylate copolymer.

7. The agent according to claims 1 to 6 wherein the basic polymer is contained in the matrix or in the adhesive layer.

8. The agent according to claims 1 to 7 wherein the basic polymer in the matrix or in the adhesive layer contains a tackifier.

9. The agent according to claims 1 to 8 wherein the tackifier contains resins and/or neutral polyacrylates.

10. The agent according to claims 1 to 9, characterized in that it contains 1 to 20% by wt. of tackifier.

11. The agent according to claims 1 to 10, characterized in that it contains 2 to 10% by wt. of tackifier.

12. The agent according to claims 1 to 11 wherein the ergolin compound is lisuride or proterguride.

13. Use of an agent according to claims 1 to 12 for the prophylaxis and treatment of diseases that can be treated using dopamine.

14. Use of an agent according to claims 1 to 13 for the treatment of Parkinson's disease, for the treatment and prevention of restless legs syndrome, premenstrual syndrome, as well as for lactation inhibition and migraine prophylaxis.
